(19) United States
(12) Patent Application Publication
(10) Pub. No.: US 2026/0151134 A1
(43) Pub. Date: Jun. 4, 2026

(54) LEFT ATRIAL APPENDAGE CLOSURE DEVICES

(71) Applicant: BOSTON SCIENTIFIC SCIMED, INC., MAPLE GROVE, MN (US)

(72) Inventors: Patrick Willoughby, Shoreview, MN (US); Scott Andrew McCullough, Plymouth, MN (US); Troy Anthony Giese, Blaine, MN (US); Philip Andrew Litecky, Forest Lake, MN (US)

(73) Assignee: BOSTON SCIENTIFIC SCIMED, INC., MAPLE GROVE, MN (US)

(21) Appl. No.: 19/360,399

(22) Filed: Oct. 16, 2025

Related U.S. Application Data
(60) Provisional application No. 63/708,520, filed on Oct. 17, 2024.

(51) Int. Cl. A61B 17/12; A61B 17/00

(52) U.S. Cl. CPC A61B 17/1219; A61B 17/12031; A61B 17/12122; A61B 2017/00004; A61B 2017/0084; A61B 2017/00867; A61B 2017/00893; A61B 2017/00942

(57) ABSTRACT

A left atrial appendage closure (LAAC) device may be adapted for occluding a patient's left atrial appendage (LAA). The LAAC device includes a shape memory polymer foam body that is adapted to expand from a crimped configuration for delivery to an expanded configuration after delivery. The shape memory polymer foam body includes a first portion that is adapted to have a first foam characteristic and a second portion that is adapted to have a second foam characteristic that is different from the first foam characteristic. The first and second foam characteristics may include one or more of foam expansion rate, foam stiffness and foam density, for example.

### CROSS REFERENCE TO RELATED APPLICATIONS

This application claims the benefit of priority of U.S. Provisional Application No. 63/708,520 filed Oct. 17, 2024, the entire disclosure of which is hereby incorporated by reference.

### TECHNICAL FIELD

The disclosure relates generally to medical devices and more particularly to medical devices that incorporate a shape memory foam component.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted for occluding a patient's left atrial appendage (LAA). The LAAC device includes a shape memory polymer foam body that is adapted to expand from a crimped configuration for delivery to an expanded configuration after delivery. The shape memory polymer foam body includes a first portion that is adapted to have a first foam characteristic and a second portion that is adapted to have a second foam characteristic that is different from the first foam characteristic.

Alternatively or additionally, the first foam characteristic may include a first expansion rate and the second foam characteristic may include a second expansion rate.

Alternatively or additionally, the first portion may have a slower expansion rate than the second portion.

Alternatively or additionally, the first portion may correspond to a distal portion of the shape memory polymer foam body and the second portion may correspond to a proximal portion of the shape memory polymer foam body.

Alternatively or additionally, the shape memory polymer foam body may further include an intermediate portion that is disposed between the first portion and the second portion and that has a third expansion rate that is intermediate of the first portion and of the second portion.

Alternatively or additionally, the first foam characteristic may include a first stiffness and the second foam characteristic may include a second stiffness.

Alternatively or additionally, the first portion may include a core structure of the shape memory polymer foam body and the second portion may include a sealing bumper surrounding the core structure.

Alternatively or additionally, the core structure may have a higher stiffness than the outer portion.

Alternatively or additionally, the first foam characteristic may include a first foam density and the second foam characteristic may include a second foam density.

Another example may be found in a left atrial appendage closure (LAAC) device that is adapted for occluding a patient's left atrial appendage (LAA). The LAAC device includes a shape memory polymer foam body that is adapted to expand from a crimped configuration for delivery to an expanded configuration after delivery. The shape memory polymer foam body includes an inner portion that is adapted to have a dense foam cell structure and an outer portion that is adapted to have a sparse foam cell structure, the outer portion extending over at least part of the inner portion.

Alternatively or additionally, the inner portion may include an anchor structure.

Alternatively or additionally, the inner portion may include a step-wise diameter anchor structure.

Alternatively or additionally, the inner portion may include one or more arms extending radially outwardly from a central point.

Alternatively or additionally, the one or more arms may be adapted to be folded when the shape memory polymer foam body is in the crimped configuration.

Alternatively or additionally, the one or more arms may provide an outward force when the shape memory polymer foam body expands into the expanded configuration.

Alternatively or additionally, the outward force may include a radially extending force.

Alternatively or additionally, the outward force may include a tangentially extending force.

Another example may be found in a left atrial appendage closure (LAAC) device that is adapted for occluding a patient's left atrial appendage (LAA). The LAAC device includes a shape memory polymer foam body that is adapted to expand from a crimped configuration for delivery to an expanded configuration after delivery. The shape memory polymer foam body includes a distal portion that is adapted to have a first expansion rate and a proximal portion that is adapted to have a second expansion rate different from the first expansion rate.

Alternatively or additionally, the first expansion rate may be slower than the second expansion rate.

Alternatively or additionally, the proximal portion may include a proximal face that may be adapted to accommodate a controlled release of an agent from the proximal face.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a schematic view of an illustrative LAAC device;

FIG. 3 is a schematic view of an illustrative LAAC device;

FIG. 4 is a schematic view of an illustrative LAAC device;

FIG. 5 is a schematic view of an illustrative LAAC device;

FIGS. 6A, 6B, and 6C are schematic views illustrating assembly of an illustrative LAAC device;

FIG. 6D is a schematic view of the illustrative LAAC device of FIGS. 6A, 6B and 6C, showing how an inner core of the illustrative LAAC device may fold;

FIG. 7 is a schematic view of an illustrative inner core forming part of an illustrative LAAC device, the inner core adapted to exert a tangential force on a wall of the LAA; and

FIG. 8 is a schematic view of an illustrative inner core forming part of an illustrative LAAC device, the inner core adapted to exert a radial force on a wall of the LAA.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

In some instances, a left atrial appendage closure (LAAC) device is adapted for occluding a patient's left atrial appendage (LAA). The LAAC device includes a shape memory polymer foam body that is adapted to expand from a crimped configuration for delivery to an expanded configuration after delivery. The shape memory polymer foam body includes a first portion that is adapted to have a first foam characteristic and a second portion that is adapted to have a second foam characteristic that is different from the first foam characteristic.

In some cases, the first foam characteristic may include a first expansion rate and the second foam characteristic may include a second expansion rate. As an example, the first portion may have a slower expansion rate than the second portion. In some cases, the first portion may correspond to a distal portion of the shape memory polymer foam body and the second portion may correspond to a proximal portion of the shape memory polymer foam body. In some cases, the shape memory polymer foam body may further include an intermediate region that is disposed between the first portion and the second portion and that has a third expansion rate that is intermediate of the first portion and of the second portion.

In some cases, the first foam characteristic may include a first stiffness and the second foam characteristic may include a second stiffness. As an example, the first portion may include a core structure of the shape memory polymer foam body and the second portion may include an outer portion sealing bumper surrounding the core structure. In some cases, the core structure may have a higher stiffness than the outer portion. In some cases, the first foam characteristic may include a first foam density and the second foam characteristic may include a second foam density.

In some instances, a left atrial appendage closure (LAAC) device may be adapted for occluding a patient's left atrial appendage (LAA). The LAAC device includes a shape memory polymer foam body that is adapted to expand from a crimped configuration for delivery to an expanded configuration after delivery. The shape memory polymer foam body includes an inner portion that is adapted to have a dense foam cell structure and an outer portion that is adapted to have a sparse foam cell structure. The outer portion extends over at least part of the inner portion.

In some cases, the inner portion may include an anchor structure. In some cases, the inner portion may include a stepped anchor structure. In some cases, the inner portion may include one or more arms extending radially outwardly from a central point. The one or more arms may be adapted to be folded when the shape memory polymer foam body is in the crimped configuration, for example. In some cases, the one or more arms may provide an outward force when the shape memory polymer foam body expands into the expanded configuration. In some cases, the outward force may include a radially extending force. In some cases, the outward force may include a tangentially extending force.

In some instances, a left atrial appendage closure (LAAC) device is adapted for occluding a patient's left atrial appendage (LAA). The LAAC device includes a shape memory polymer foam body that is adapted to expand from a crimped configuration for delivery to an expanded configuration after delivery. The shape memory polymer foam body includes a distal portion that is adapted to have a first expansion rate and a proximal portion that is adapted to have a second expansion rate different from the first expansion rate. In some cases, the first expansion rate may be slower than the second expansion rate. In some cases, the proximal portion may include a proximal face that is adapted to accommodate a controlled release of a chemical agent from the proximal face.

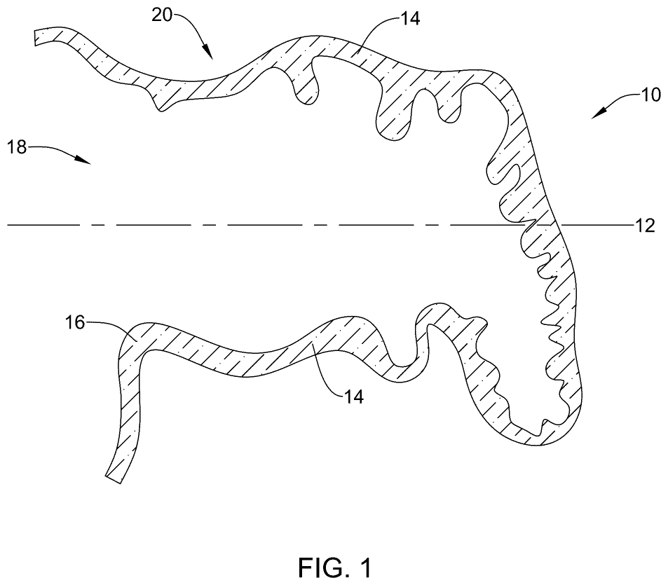

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

LAAC devices may be made from foam materials such as shape memory polymer foams that have a compressed configuration and an expanded configuration. In some cases, LAAC devices may be made from two or more distinct pieces or sections of shape memory polymer foam, where each of the two or more distinct pieces or sections of shape memory polymer foam may have different properties. The composition of a shape memory polymer foam LAAC device may vary from distal end to proximal end, for example. The composition of a shape memory polymer foam LAAC device may vary radially within the LAAC device.

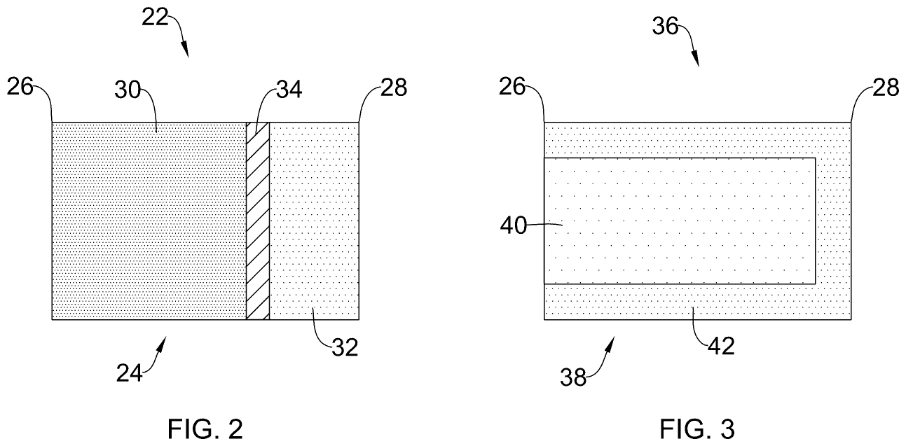

FIG. 2 is a schematic view of an illustrative LAAC device 22 in which the composition of the shape memory polymer foam varies axially, i.e., distally to proximally. The LAAC device 22 includes a shape memory polymer foam body 24 that extends from a first end 26 to a second end 28. In some cases, the first end 26 may be considered as being a distal end while the second end 28 may be considered as being a proximal end. In some cases, the first end 26 may be considered as being a proximal end while the second end 28 may be considered as being a distal end. It will be appreciated that distal and proximal are defined by the orientation in which the LAAC device 22 is implanted. The shape memory polymer foam body 24 includes a first portion 30 that extends to the first end 26 and a second portion 32 that extends to the second end 28. In some cases, as shown, the shape memory polymer foam body 24 may include an intermediate portion 34 that is positioned intermediate, or between, the first portion 30 and the second portion 32. In some cases, the first portion 30 may be adapted to have a first foam characteristic and the second portion 32 may be adapted to have a second foam characteristic that is different from the first foam characteristic. The intermediate portion 34, if present, may have a third foam characteristic that is intermediate the first foam characteristic and the second foam characteristic.

In some cases, the first foam characteristic of the first portion 30 may be a first expansion rate and the second foam characteristic of the second portion 32 may be a second expansion rate. As an example, a distal region of the LAAC device 22, which may be either the first portion 30 or the second portion 32, may have an expansion rate that is slower than that of the proximal region. In some cases, having a slower expansion rate in the distal region can help to prevent or slow down expansion of the LAAC device 22 while the LAAC device 22 remains at least partially within a delivery system. In some cases, having a faster expansion rate in a proximal region can help the LAAC device 22 to expand rapidly after delivery and thus can immediately begin to seal off the LAA 10. The intermediate portion 34 may have an expansion rate that is faster than that of the distal region and slower than that of the proximal region.

FIG. 3 is a schematic view of an illustrative LAAC device 36 in which the composition of the shape memory polymer foam varies radially, i.e., inner to outer. The LAAC device 36 includes a shape memory polymer foam body 38 that extends from a first end 26 to a second end 28. In some cases, the first end 26 may be considered as being a distal end while the second end 28 may be considered as being a proximal end. In some cases, the first end 26 may be considered as being a proximal end while the second end 28 may be considered as being a distal end. It will be appreciated that distal and proximal are defined by the orientation in which the LAAC device 36 is implanted. The shape memory polymer foam body 38 includes an inner portion 40 and an outer portion 42. In some cases, the inner portion 40 extends from the first end 26 towards the second end 28 but does not entirely extend to the second end 28. As an example, the inner portion 40 may extend about seventy percent or more, about eighty percent or more, or about ninety percent or more of the way from the first end 26 to the second end 28. In this way, the outer portion 42 at least partially surrounds the inner portion 40. In some cases, the inner portion 40 may be adapted to have a first foam characteristic and the outer portion 42 may be adapted to have a second foam characteristic that is different from the first foam characteristic.

In some cases, the first foam characteristic of the inner portion 40 may be a first stiffness and the second foam characteristic of the outer portion 42 may be a second stiffness. In some cases, the inner portion 40 may have a greater stiffness than the outer portion 42. In some cases, the inner portion 40 may expand into the outer portion 42, thereby causing the LAAC device 36 to expand and push into the anatomy with enough force to provide at least some anchoring force. In some cases, the inner portion 40 may push the outer portion 42 into contact with the interior of the LAA 10, causing the outer portion 42 to compress.

In some cases, the first foam characteristic of the inner portion 40 may be a first density and the second foam characteristic of the outer portion 42 may be a second density. In some cases, the inner portion 40 may have a dense foam cell structure while the outer portion 42 may have a sparse foam cell structure. As a result, the inner portion 40 may be stronger and harder, with less resiliency, while the outer portion 42 may be softer, with more resiliency. In some cases, the outer portion 42 may act as a bumper that surrounds the inner portion 40, particularly a part of the outer portion 42 that extends across an end of the inner portion 40. In some cases, the outer portion 42 may be adapted to absorb blood and thus convert to tissue faster than the inner portion 40 does.

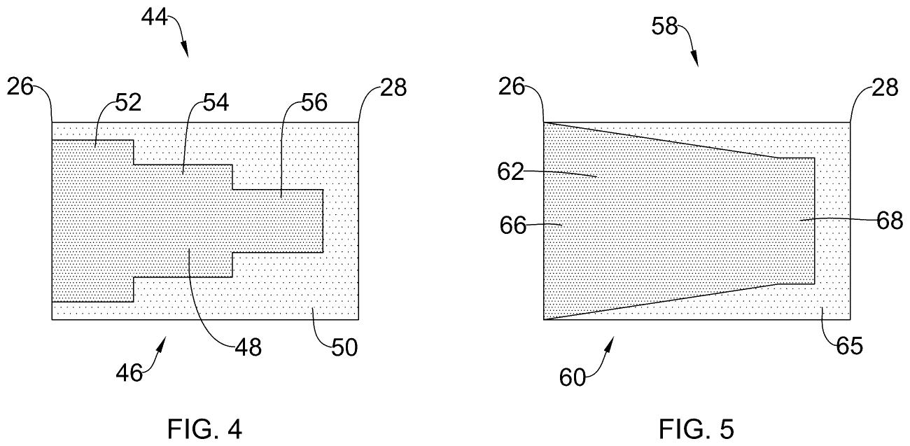

In some cases, as shown, the inner portion 40 may have a cylindrical or at least substantially cylindrical profile. In some cases, the inner portion 40 may include one or more step-wise changes in diameter, and/or one or more tapered changes in diameter. FIG. 4 provides an example of step-wise diameter changes in a portion of a shape memory polymer foam body. FIG. 4 is a schematic view of an illustrative LAAC device 44. The illustrative LAAC device 44 includes a shape memory polymer foam body 46 that extends from a first end 26 to a second end 28. In some cases, the first end 26 may be considered as being a distal end while the second end 28 may be considered as being a proximal end. In some cases, the first end 26 may be considered as being a proximal end while the second end 28 may be considered as being a distal end. It will be appreciated that distal and proximal are defined by the orientation in which the LAAC device 44 is implanted. The shape memory polymer foam body 46 includes an inner portion 48 and an outer portion 50. In some cases, the inner portion 48 extends from the first end 26 towards the second end 28 but does not entirely extend to the second end 28. As an example, the inner portion 48 may extend about seventy percent or more, about eighty percent or more, or about ninety percent or more of the way from the first end 26 to the second end 28. In this way, the outer portion 50 at least partially surrounds the inner portion 48. In some cases, the inner portion 48 may be adapted to have a first foam characteristic and the outer portion 50 may be adapted to have a second foam characteristic that is different from the first foam characteristic.

As shown in FIG. 4, the inner portion 48 includes a first diameter segment 52, a second diameter segment 54 having a diameter that is less than that of the first diameter segment 52, and a third diameter segment 56 having a diameter that is less than that of the second diameter segment 54. While three distinct segments 52, 54 and 56 are shown, this is merely illustrative. In some cases, the inner portion 48 may only have two diameter changes. In some cases, the inner portion 48 may have four or more diameter changes. In some cases, having the first diameter segment 52, the largest diameter segment, at the distal end can provide additional interference and more anchoring deeper in the anatomy. Having the third diameter segment 56, the smallest diameter segment, at the proximal end can mean less anchor force and better conformability and sealing relative to the LAA 10. In some cases, having the smallest diameter segment at the distal end and the largest diameter segment at the proximal end may provide a more uniform force distribution along the length of the LAAC device 44.

In some cases, the first foam characteristic of the inner portion 48 may be a first stiffness and the second foam characteristic of the outer portion 50 may be a second stiffness. In some cases, the inner portion 48 may have a greater stiffness than the outer portion 50. In some cases, the inner portion 48 may expand into the outer portion 50 and the anatomy with enough force to provide at least some anchoring force. In some cases, the first foam characteristic of the inner portion 48 may be a first density and the second foam characteristic of the outer portion 50 may be a second density. In some cases, the inner portion 48 may have a dense foam cell structure while the outer portion 50 may have a sparse foam cell structure. As a result, the inner portion 48 may be stronger and harder, with less resiliency, while the outer portion 50 may be softer, with more resiliency. In some cases, the outer portion 50 may act as a bumper that surrounds the inner portion 48, particularly a part of the outer portion 50 that extends across an end of the inner portion 48. In some cases, the outer portion 50 may be adapted to absorb blood and thus convert to tissue faster than the inner portion 48 does.

FIG. 5 is a schematic view of an illustrative LAAC device 58. The illustrative LAAC device 58 includes a shape memory polymer foam body 60 that extends from a first end 26 to a second end 28. In some cases, the first end 26 may be considered as being a distal end while the second end 28 may be considered as being a proximal end. In some cases, the first end 26 may be considered as being a proximal end while the second end 28 may be considered as being a distal end. It will be appreciated that distal and proximal are defined by the orientation in which the LAAC device 58 is implanted. The shape memory polymer foam body 60 includes an inner portion 62 and an outer portion 64. In some cases, the inner portion 62 extends from the first end 26 towards the second end 28 but does not entirely extend to the second end 28. As an example, the inner portion 62 may extend about seventy percent or more, about eighty percent or more, or about ninety percent or more of the way from the first end 26 to the second end 28. In this way, the outer portion 64 at least partially surrounds the inner portion 62. In some cases, the inner portion 62 may be adapted to have a first foam characteristic and the outer portion 64 may be adapted to have a second foam characteristic that is different from the first foam characteristic.

As shown in FIG. 5, the inner portion 62 includes a tapered segment 66 and a constant diameter segment 68. While a single tapered segment 66 and a single constant diameter segment 68 are shown, it will be appreciated that the inner portion 62 may include two or more tapered segments and/or may include two or more constant diameter segments. The inner portion 62 may include one or more tapered segments and one or more step-wise changes in diameter. In some cases, having the first diameter portion at the distal end can provide additional interference and more anchoring deeper in the anatomy. Having the smallest diameter segment at the proximal end can mean less anchor force and better conformability and sealing relative to the LAA 10. In some cases, having the smallest diameter segment at the distal end and the largest diameter segment at the proximal end may provide a more uniform force distribution along the length of the LAAC device 58.

In some cases, the first foam characteristic of the inner portion 62 may be a first stiffness and the second foam characteristic of the outer portion 64 may be a second stiffness. In some cases, the inner portion 62 may have a greater stiffness than the outer portion 64. In some cases, the inner portion 62 may expand into the anatomy with enough force to provide at least some anchoring force. In some cases, the first foam characteristic of the inner portion 62 may be a first density and the second foam characteristic of the outer portion 64 may be a second density. In some cases, the inner portion 62 may have a dense foam cell structure while the outer portion 64 may have a sparse foam cell structure. As a result, the inner portion 62 may be stronger and harder, with less resiliency, while the outer portion 64 may be softer, with more resiliency. In some cases, the outer portion 64 may act as a bumper that surrounds the inner portion 62, particularly a part of the outer portion 64 that extends across an end of the inner portion 62. In some cases, the outer portion 64 may be adapted to absorb blood and thus convert to tissue faster than the inner portion 62 does.

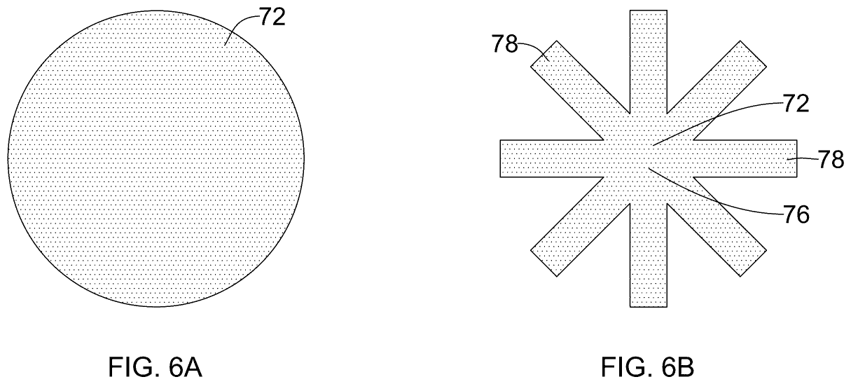

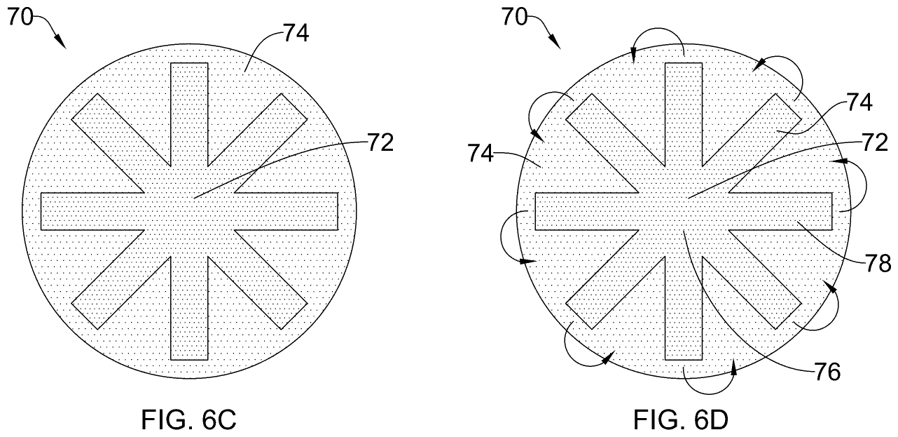

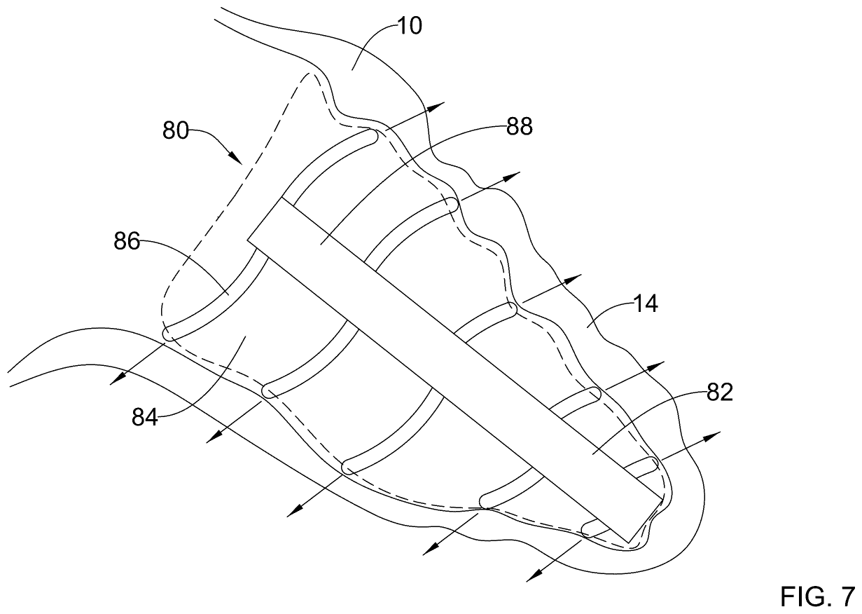

FIGS. 6A, 6B and 6C schematically show a process for forming an LAAC device 70 that includes an inner portion 72 and an outer portion 74. In FIG. 6A, the inner portion 72 begins as a cylindrical shape. The inner portion 72 is formed of a shape memory polymer foam having a dense foam cell structure that provides the inner portion 72 with enough stiffness to resist external forces and to allow for user-applied forces when deploying the LAAC device 70. As shown in FIG. 6B, a fraction of the inner portion 72 is removed, leaving a central segment 76 and a number of arms 78 that extend radially outwardly from the central segment 76 when in an expanded configuration, as shown. While a total of eight arms 78 are shown, this is merely illustrative, as the inner portion 72, once processed, may have any number of arms 78, including seven or fewer arms 78 or nine or more arms 78. As an example, the arms 78 may be formed by cutting away the material originally present between neighboring arms 78. In some cases, removing material from the inner portion 72 results in a better overall compressibility for the LAAC device 70 when the LAAC device 70 is crimped for delivery.

In FIG. 6C, the outer portion 74 has been formed around the inner portion 72, such that the central segment 76 and each of the radially extending arms 78 are embedded within the outer portion 74. In some cases, the arms 78 may provide an outward force when the LAAC device 70 expands from a crimped configuration to an expanded configuration. The outward force may be a radially directed force. The outward force may be a tangentially directed force. In some cases, as shown in FIG. 6D, the arms 78 may be folded over when the LAAC device 70 is compressed or crimped into a delivery configuration. This may impact how the LAAC device 70 expands once the LAAC device 70 is allowed to expand.

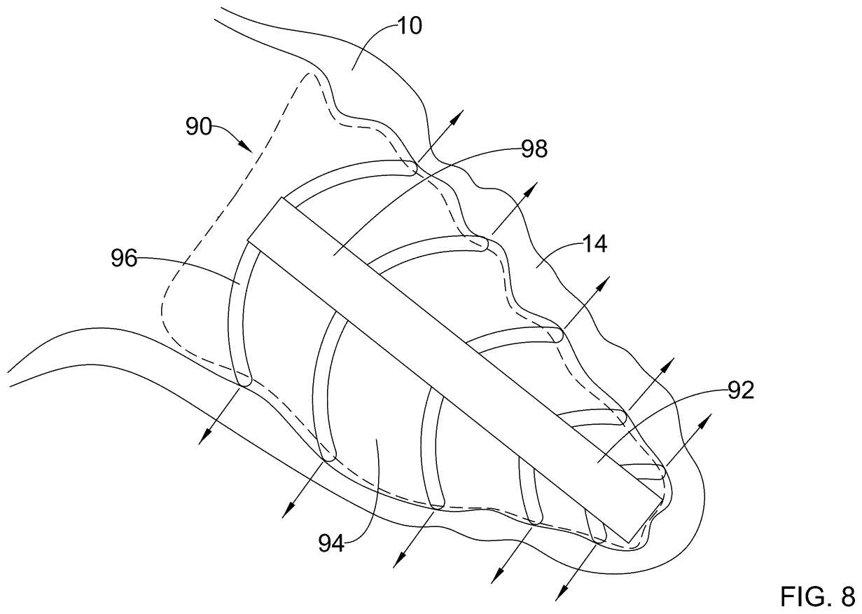

FIG. 7 shows an LAAC device 80 disposed within the LAA 10. As shown, the LAAC device 80 includes an inner portion 82 and an outer portion 84. In some cases, the inner portion 82 may include arms 86 that extend tangentially from a central region 88 such that when the LAAC device 80 expands, the arms 86 exert a tangential force on the wall 14 of the LAA 10. In some cases, the inner portion 82, including the arms 86, is formed and then the outer portion 84 may be formed around the inner portion 82. The arms 86 may be molded as part of the inner portion 82, or portions of the inner portion 82 may be removed to leave the arms 86 extending from the central region 88. The inner portion 82 may include any number of arms 86, and the arms 86 may be arranged such that there are multiple rows of arms 86, with two or more arms 86 in each of the rows. In some cases, there may be as many as ten rows of arms 86. An axial distance between rows of the arms 86 may be influenced by how many rows of arms 86 there are.

FIG. 8 shows an LAAC device 90 disposed within the LAA 10. As shown, the LAAC device 90 includes an inner portion 92 and an outer portion 94. In some cases, the inner portion 92 may include arms 96 that extend tangentially from a central region 98 such that when the LAAC device 90 expands, the arms 96 exert a radial force on the wall 14 of the LAA 10. In some cases, the inner portion 92, including the arms 96, is formed and then the outer portion 94 may be formed around the inner portion 92. The arms 96 may be molded as part of the inner portion 92, or portions of the inner portion 92 may be removed to leave the arms 96 extending from the central region 98. The inner portion 92 may include any number of arms 96, and the arms 96 may be arranged such that there are multiple rows of arms 96, with two or more arms 96 in each of the rows. In some cases, there may be as many as ten rows of arms 96. An axial distance between rows of the arms 96 may be influenced by how many rows of arms 96 there are.

In some cases, parts of the shape memory polymer foam components described herein may include a porosity that is tuned to provide a controlled and extended release of an agent contained within the resulting process. For example, a large number of smaller pores could be coated with an agent elution layer. Elution timing would depend on the coating release rate plus the transfer time out of the foam pore tortuosity. Since the foam has a slow degradation rate, an agent may also be contained in mostly closed pores in the foam. In some cases, foam degradation occurs first on the thin windows in between the foam struts, which may pop the bubbles and release the agent. As an example, the released agent could be a drug such as an anti-coagulant, an anti-inflammatory or an, antibiotic). The released agent could be an imaging modifier such as e.g. fluoro contrast, MRI contrast, or an echogenic substance.

In some cases, parts of the shape memory polymer foam components described herein, particularly outside surfaces intended to contact tissue may include a porosity and/or stiffness that may be adjusted to provide larger openings with stiffer characteristics. This may allow for faster expansion of the outer surface and faster ingress of blood into the device. A stiffer structure may provide more anchoring capability against/into tissue. The same properties could also be formed into a disc or cylindrical core in the foam that would provide the main structure and allow blood to more quickly fill into the inside of the device then with a consistent smaller pored structure.

In some cases, it may be possible to have a low density interior that is encapsulated within a higher density, higher stiffness exterior layer to decrease total mass and crimped profile while maintaining large volume. Outer and inner materials may differ by one or more of chemical composition, pore size, and thickness, among others. In some cases, two different foam components may be joined together with a barrier layer disposed between the two different foam components. The proximal foam component may be selected to optimize sealing and conformability to the anatomy. The distal foam structure could be optimized to allow for integration of gels or adhesives, or to allow for riskier procedures to be performed behind the foam.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D, L-lactide, poly(beta-hydroxybutyrate), polygluconate including poly-D-gluconate, poly-L-gluconate, poly-D, L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha, omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine-and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding a patient's left atrial appendage (LAA), the LAAC device comprising:
a shape memory polymer foam body adapted to expand from a crimped configuration for delivery to an expanded configuration after delivery, the shape memory polymer foam body including:
a first portion adapted to have a first foam characteristic; and
a second portion adapted to have a second foam characteristic that is different from the first foam characteristic.

2. The LAAC device of claim 1, wherein the first foam characteristic comprises a first expansion rate and the second foam characteristic comprises a second expansion rate.

3. The LAAC device of claim 2, wherein the first portion has a slower expansion rate than the second portion.

4. The LAAC device of claim 3, wherein the first portion corresponds to a distal portion of the shape memory polymer foam body and the second portion corresponds to a proximal portion of the shape memory polymer foam body.

5. The LAAC device of claim 2, wherein the shape memory polymer foam body further comprises an intermediate portion disposed between the first portion and the second portion, the intermediate portion having a third expansion rate that is intermediate of the first portion and of the second portion.

6. The LAAC device of claim 1, wherein the first foam characteristic comprises a first stiffness and the second foam characteristic comprises a second stiffness.

7. The LAAC device of claim 6, wherein the first portion comprises a core structure of the shape memory polymer foam body and the second portion comprises a sealing bumper surrounding the core structure.

8. The LAAC device of claim 7, wherein the core structure has a higher stiffness than the outer portion.

9. The LAAC device of claim 1, wherein the first foam characteristic comprises a first foam density and the second foam characteristic comprises a second foam density.

10. A left atrial appendage closure (LAAC) device adapted for occluding a patient's left atrial appendage (LAA), the LAAC device comprising:
a shape memory polymer foam body adapted to expand from a crimped configuration for delivery to an expanded configuration after delivery, the shape memory polymer foam body including:
an inner portion adapted to have a dense foam cell structure; and
an outer portion adapted to have a sparse foam cell structure, the outer portion extending over at least part of the inner portion.

11. The LAAC device of claim 10, wherein the inner portion comprises an anchor structure.

12. The LAAC device of claim 11, wherein the inner portion comprises a step-wise diameter anchor structure.

13. The LAAC device of claim 10, wherein the inner portion comprises one or more arms extending radially outwardly from a central point.

14. The LAAC device of claim 13, wherein the one or more arms are adapted to be folded when the shape memory polymer foam body is in the crimped configuration.

15. The LAAC device of claim 14, wherein the one or more arms provide an outward force when the shape memory polymer foam body expands into the expanded configuration.

16. The LAAC device of claim 15, wherein the outward force comprises a radially extending force.

17. The LAAC device of claim 15, wherein the outward force comprises a tangentially extending force.

18. A left atrial appendage closure (LAAC) device adapted for occluding a patient's left atrial appendage (LAA), the LAAC device comprising:
a shape memory polymer foam body adapted to expand from a crimped configuration for delivery to an expanded configuration after delivery, the shape memory polymer foam body including:
a distal portion adapted to have a first expansion rate; and
a proximal portion adapted to have a second expansion rate different from the first expansion rate.

19. The LAAC device of claim 18, wherein the first expansion rate is slower than the second expansion rate.

20. The LAAC device of claim 18, wherein the proximal portion includes a proximal face that is adapted to accommodate a controlled release of an agent from the proximal face.
